(19) United States
(12) Patent Application Publication
(10) Pub. No.: US 2004/0034285 A1
(43) Pub. Date: Feb. 19, 2004

(54) Body movement analysis system and body movement analysis method

(76) Inventor: Takeshi Sahashi, Arajyuku Kawagoe Saitama (JP)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/415,777

(22) PCT Filed: Sep. 27, 2001
(86) PCT No.: PCT/JP01/08447

(30) Foreign Application Priority Data
Oct. 31, 2000 (JP) 2000-332793

(51) Int. Cl. A61B 5/00

(52) U.S. Cl. 600/300

(57) ABSTRACT

A system and method are provided capable of analyzing body movement using time series data obtained continuously using a sensor measuring biological information other than body movement.

A body analysis system having measuring means for continuously measuring biological information from a body to obtain time series data, and extraction means for extracting low frequency components of a prescribed frequency or lower from the time series data as body movement waveform data, and a body movement analysis method having measuring steps and extraction steps carried out using this body movement analysis system are provided. A low-frequency component generated from body movement is included in the time series data obtained through continuous measurement of biological information from the body. Conventionally, this low frequency component was eliminated as noise. The body movement analysis system and body movement analysis method of the present invention is capable of obtaining body movement waveform data expressing body movement by extracting low frequency components less than or equal to a prescribed frequency from the time series data.

### TECHNICAL FIELD

The present invention relates to a body movement analysis system and body movement analysis method.

### BACKGROUND ART

In the case of medical treatment, conventionally, biological information has been measured for a body continuously using a sensor such as a heartbeat monitor, respiration monitor or pulse oximeter and time series data is then obtained. Time series data acquired using a sensor is then usually comprehended as a waveform and displayed.

These sensors usually aim at acquiring biological information for specific organs or parts of the body. For example, the purpose of a heartbeat monitor is to extract biological information for an organ referred to as a heart and as such measures movement of a heart as changes in potential. Time series data for these changes in potential then makes up an electrocardiogram. In this specification, a heartbeat monitor refers to a monitor for sensing a heartbeat from this kind of movement of a heart.

It is therefore possible to understand biological information for specific organs or parts using this kind of continuously obtained time series data. A frequency band peculiar to this specific organ or part exists for the biological information for the specific organ or part being a target for measurement using the sensors. It is therefore demanded that the sensors reliably measure time series data for this frequency band. For example, when obtaining an electrocardiogram, in the case of a persons body, it is necessary that time series data for a frequency band of roughly 1 to 25 Hertz be measured. An example of a block diagram for a system used conventionally in this kind of measurement is shown in FIG. 10.

This kind of sensor often also acquires data for frequencies other than the target band. According to the research of this inventor, amplitudes for frequency bands less than or equal to a prescribed value often represent body movement. It is therefore also possible to understand body movement by observing amplitudes in low frequency bands which were formerly understood as being noise in the related art. In doing so, it is therefore possible to observe body movement with comparatively good precision using a sensor. It is therefore possible to alleviate the load placed on supervisors such as doctors and nurses.

It is therefore the object of the present invention to provide a system and method that enable analysis of body movement using time series data obtained through continuous measurement of biological information from a body.

### DISCLOSURE OF THE INVENTION

The present application therefore considered the utilization of low-frequency components formerly eliminated as noise from time series data continuously obtained by a sensor such as a heartbeat monitor or respiration monitor for measuring specific biological information of a patient. Namely, low-frequency components that were as a matter of preference eliminated as noise from time series data constituted by cardiogram and respiratory waveforms etc. in the related art are utilized and these low-frequency components are extracted as data constituting body movement.

The present inventor therefore invented a body movement analysis system comprising measuring means for continuously measuring biological information from a body and obtaining time series data, and extraction means for extracting low frequency components of a prescribed frequency or lower from the time series data as body movement waveform data.

The body movement analysis system of the present invention continuously measures biological information from a body and obtains time series data. Body movement waveform data is then extracted from the time series data obtained in this manner.

This time series data obtained by continuously measuring biological information from a body can then be made into an observable waveform. For example, time series data obtained using a heartbeat monitor can be made to form an electrocardiogram.

Here, time series data does not have to be time series data obtained by continuously measuring biological information from a body in order to measure just body movement. For example, it is possible to use time series data obtained by measuring biological information for specific organs or parts of a body such as with heartbeat monitors, respiration monitors or pulse oximeters. Further, here, body movement does not mean movement peculiar to specific organs of parts of a body such as for a specific organ of a body such as, for example, a heart or respiratory organ. Body movement means substantial movements of the body itself.

In time series data obtained by continuously measuring biological information of a specific organ of a body, a frequency component of a frequency band peculiar to this specific organ is included as a main component. However, other frequency components, in particular, low frequency components resulting from body movement, are also included. This low frequency component is viewed as noise from the point of view of the biological information comprehended from this time series data but is also a component for which body movement waveform data can be extracted.

The body movement analysis system of the present invention therefore utilizes time series data obtained continuously by continuously measuring biological information for specific organs or parts of a body as time series data for extracting body movement. The low frequency component which has conventionally been eliminated as noise is then extracted from this time series data as body movement waveform data.

Extraction of the low frequency component as body movement waveform data does not necessarily mean the extraction of the low-frequency component in the form of an observable body movement waveform. The data may also be a frequency obtained using Fourier analysis and amplitude data thereof. The data may be digital data or analog data. To summarize, the meaning of body movement waveform data refers to data which is capable of making up an observable body movement waveform using an extracted low-frequency component.

The present inventor therefore invented a body movement analysis method comprising a measurement step for continuously measuring biological information from a body and obtaining time series data, and an extraction step for extracting low frequency components of a prescribed frequency or lower from the time series data as body movement waveform data.

The body movement analysis method of the present invention continuously measures biological information from a body and obtains time series data. Body movement waveform data is then extracted from the time series data obtained in this manner.

This time series data obtained by continuously measuring biological information from a body can then be made into an observable waveform. For example, time series data obtained using a heartbeat monitor can be made to form an electrocardiogram.

Here, time series data also does not have to be time series data obtained by continuously measuring biological information from a body in order to measure just body movement. For example, time series data obtained by measuring biological information for specific organs or parts of a body such as with heartbeat monitors, respiration monitors or pulse oximeters may also be used. Further, here, body movement does not mean movement peculiar to specific organs of parts of a body such as for a specific organ of a body such as, for example, a heart or respiratory organ. Body movement means substantial movements of the body itself.

In time series data obtained by continuously measuring biological information of a specific organ of a body, a frequency component of a frequency band peculiar to this specific organ is included as a main component. However, other frequency components, in particular, low frequency components resulting from body movement, are also included. This low frequency component is viewed as noise from the point of view of the biological information comprehended from this time series data but is also a component for which body movement waveform data can be extracted.

The body movement analysis method of the present invention therefore utilizes time series data obtained continuously by continuously measuring biological information for specific organs or parts of a body as time series data for extracting body movement. The low frequency component which has conventionally been eliminated as noise is then extracted from this time series data as body movement waveform data.

Here, extraction of the low frequency component as body movement waveform data does not necessarily mean the extraction of the low-frequency component in the form of an observable body movement waveform. This means that it is also possible to make an observable body movement waveform from the extracted low-frequency waveform similarly in that it is possible to make an observable waveform from the time series data.

### BRIEF DESCRIPTION OF THE DRAWINGS

FIG. 1 is a view showing an outline of a body movement analysis system of the present invention.

FIG. 2 is a view showing an electrocardiogram.

FIG. 3 is a view showing a respiratory waveform.

FIG. 4(A) is a view showing a waveform for a low-frequency component of one Hertz or less extracted from the cardiograph of FIG. 2. FIG. 4(B) is a view of a waveform for a low frequency component of 0.5 Hertz or less extracted from the cardiograph of FIG. 2.

FIG. 5(A) is a view showing a waveform for a low-frequency component of one Hertz or less extracted from the respiratory waveform of FIG. 3. FIG. 5(B) is a view of a waveform for a low frequency component of 0.5 Hertz or less extracted from the respiratory waveform of FIG. 3.

FIG. 6(A), (B), (C) and (D) are views showing waveforms all occurring in the same timeband. FIG. 6(A) is a view showing a respiratory waveform and FIG. 6(B) is a view showing a waveform for a low-frequency component of 0.5 Hertz or less extracted from the respiratory waveform of FIG. 6(A). FIG. 6(C) is a view showing a cardiograph and FIG. 6(D) is a view showing a waveform for a low-frequency component of 0.5 Hertz or less extracted from the cardiograph of FIG. 6(C).

FIG. 7 is a view showing a waveform extracted for a low-frequency component of 0.5 Hertz or less from the cardiograph with horizontal lines drawn at values of +/−200 mV.

FIG. 8 is a view showing horizontal lines drawn at values of +/−200 mV at the cardiograph.

FIG. 9 is a view illustrating an example of a method of extracting high-amplitude low-frequency components.

FIG. 10 is a view showing the outline of a system for eliminating body movement from time series data continuously obtained from a body by a sensor.

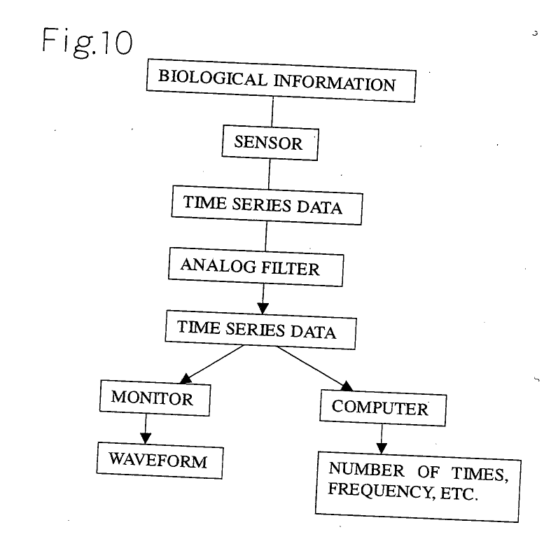

### PREFERRED EMBODIMENTS

(Embodiment of a Body Movement Analysis System)

The body movement analysis system of the present invention comprises measuring means for continuously measuring biological information from a body and obtaining time series data, and extraction means for extracting low frequency components of a prescribed frequency or lower from the time series data as body movement waveform data. The following is a description of the body movement analysis system of the present invention.

The body movement analysis system of the present invention has measuring means for continuously measuring biological information from a body and obtaining time series data.

Here, the “body” is by no means limited to the body of a person. As well as being a persons body, the body may also be a body other than that of a person such as that of a dog, cat or cow, etc.

Biological information continuously measured from the body is not particularly limited. This may be biological information for a heart measured using a heartbeat monitor, biological information for respiratory organs measured using a respiratory monitor, or biological information for an amount of red blood corpuscles flowing in veins measured using a pulse oximeter.

The obtained time series data is not particularly limited providing that this is time series data obtained by continuous measurement of biological information from the body. This kind of time series data may be, for example, time series data for changes in time of an action potential of a heart obtained by a heartbeat monitor, time series data for changes in a resistance value across two points of a respiratory organ obtained by a respiratory monitor, or time series data for changes in time of absorbance of oxygen saturation of red blood corpuscles due to pulsation of peripheral vessels obtained by a pulse oximeter. It is also possible for this data to be time series data for changes in an image measured by a video camera photographing a body.

The measuring means for continuously measuring biological information from the body and obtaining time series data is also not specifically limited. It is also possible to use measuring means according to the measured biological information and the obtained time series data. It is also possible to use well-known measuring means such as heartbeat monitors, respiration monitors or pulse oximeters.

This time series data obtained by continuously measuring biological information from these bodies can then be made into a waveform. For example, time series data obtained using a heartbeat monitor can be made to form an electrocardiogram. For example, time series data obtained using a respiration monitor can be used to form a respiration waveform.

The form of data for the time series data obtained by the measuring means is not specifically limited. Typically, time series data obtained through measurement of biological information from a body may take the form of an analog or digital electrical signal. However, the invention is by no means limited in this respect. For example, the time series data for the biological information measured by the measuring means may also be in the form of an optical signal. In this case, it is preferable to change the form of the electrical signals so that subsequent processing can be carried out easily.

Here, a heartbeat monitor or respiratory monitor is preferable as the measuring means. This is because, heartbeat monitors and respiratory monitors are typically often used, and, waveforms and cardiographs constituting time series data obtained through continuous measurement using these monitors are easy to use. This is also because heartbeat monitors and respiratory monitors are used for patients laying on a bed, and it is very necessary to obtain body movement information for these kinds of patients.

The body movement analysis system of the present invention has extraction means for extracting low frequency components of a prescribed frequency or lower from the time series data as body movement waveform data.

In this case, extraction of low frequency components of a prescribed frequency or less includes not only extraction of low frequency components of the prescribed frequency or less, but also extraction of a band of low frequency components of less than the prescribed frequency. Namely, a prescribed frequency or less includes setting of this prescribed frequency as the upper limit of a frequency band and then setting a lower limit.

A prescribed frequency can be selected taking into consideration the obtained body movement. From the point of view of diagnosis and treatment of people it is preferable for the prescribed frequency to be set at 0.5 Hertz. When a patient is resting by laying in a bed or sleeping, body movement is typically slow movement. Therefore, if time series data of less than 0.5 Hertz is extracted, then even body movement that is very slow can be captured.

As described above, the body movement of a patient when resting is slow movement. Therefore, in cases where it is considered that it is not necessary to monitor up to movement (for example, movement less than 0.05 Hertz) that is slow exceeding a certain fixed limit, it is preferable to extract a low frequency component from, for example, 0.05 to 0.5 Hertz.

If the prescribed frequency is set to be low, then body movement that is slow to this extent can be obtained, and if it is set high, fast body movement can be obtained. The prescribed frequency can therefore be set appropriately according to the condition of the body that is the target of measurement.

It is also possible to set the prescribed frequency taking into consideration the type and condition of movement being monitored when the body of a animal is taken as a target.

Further, extraction of the low-frequency component from the time series data includes the extraction of a low-frequency component having an amplitude above a prescribed amplitude. In this case, it is possible to perform processing while eliminating the high-amplitude component possessed by the biological information of the specific organ expressed by the time series data in this case.

Well-known appropriate means can be used as the extraction means for extracting low frequency components of less than the prescribed frequency from the time series data. For example, as means for extraction, an analog filter can be used when the time series data is configured from an analog electrical signal. For example, a filter configured using lumped parameter elements such as coils, capacitors and resistors etc. or an active filter employing transistors etc. can be used as the extraction means. In this case, a low pass filter that only permits a low frequency band to pass can be used. A band pass filter permitting a desired frequency band to pass can be used when extracting low frequency components of a fixed range frequency band taking the prescribed frequency as the upper limit.

Further, when the time series data is configured from a digital electrical signal, a digital filter can be employed as the extraction means and may be implemented using a computer, etc. In this case, the digital filter may be configured from a low-pass filter only allowing low-frequency bands to pass. Further, when it is wished to extract low-frequency components for a frequency band of a fixed range taking a prescribed frequency as an upper limit, the digital filter may be configured from a band pass filter allowing this frequency band to pass.

It is also possible to extract the low-frequency component using extraction means after converting time series data outputted from the measuring means to a signal format for which it is easy to extract the low-frequency component. It is also possible, for example, to convert an analog electrical signal into a digital electrical signal using an A/D converter and then extract the low-frequency component by using a digital filter on the time series data constituted by the converted digital electrical signal.

Further, when the measuring means measures the biological information so that the obtained time series data is configured from an optical signal, the low-frequency component may be extracted using an appropriate extraction means after converting the time series data constituted by the optical signal to time series data constituted by an electrical signal.

When the time series data outputted from the measuring means is faint, it is preferable to make the signal into time series data constituted by a signal of a strength for which extraction of the low-frequency component is straightforward, using an amplifier etc.

Low-frequency components extracted in this manner can then be understood as body movement waveform data. However, the low-frequency component extracted as body movement waveform data does not have to be considered in the form of an observable body movement waveform as described above. Extraction of the low-frequency component may take the form of extraction of an observable waveform or the form of data left as is for the low-frequency component capable of configuring an observable waveform. For example, when the extracted low-frequency component is data consisting of a digital electrical signal, the data for this digital electrical signal may be left as is.

Information for the body movement can then be obtained by analyzing the extracted low-frequency component. Namely, it is possible to extract the frequency and amplitude of the low-frequency waves making up the low frequency component, as well as the frequency with which the low frequency waves occur, the time for which the low frequency waves occur, and the strength of the low-frequency waves by analyzing the extracted low-frequency component.

Information for the magnitude, strength and duration etc. of the body movement can then be obtained by calculating the frequency, amplitude, frequency, strength and duration of the low frequency waves. Further, the strength of the low-frequency component is obtained by calculating the total of the absolute values of differences between values for low frequency components for each time occurring within a prescribed unit time and a base line for when there is no movement or the total for the square of the absolute values for these differences.

The body movement analysis system of the present invention preferably has low-frequency component strength calculating means for calculating strength of the low-frequency component from the low-frequency component. A computer etc. may be used as this low-frequency component strength calculating means.

The body movement analysis system of the present invention may also preferably comprise high-amplitude low frequency component strength calculating means for extracting a high-amplitude low-frequency component having a prescribed amplitude or greater from the low frequency component from the point of view of obtaining information relating to body movement of more than a fixed size and calculating at least one or more of strength, frequency, and duration of the high-amplitude low-frequency component.

A computer may be employed as the high-amplitude low frequency component strength calculating means for extracting a high-amplitude low-frequency component having a prescribed amplitude or greater from the extracted low frequency component and calculating strength, frequency, and duration etc. of the high-amplitude low-frequency component. It is then preferable to employ a computer from the point of view of processing and storing strength, frequency and duration, etc.

The amplitude expresses the magnitude of the body movement. Data for body movement waveforms for body movement having greater than a fixed magnitude can then be extracted by extracting high-amplitude low-frequency components having amplitudes of greater than a prescribed amplitude. The prescribed amplitude can then be set taking into consideration the desired amount of body movement and the provided time series data. For example, even in the case of a patient that is a person at rest laying on a bed, when the time series data is a respiratory waveform, it is preferable to extract amplitudes of roughly 1.5 times the average amplitude of the respiratory waveform.

The time for which each high-amplitude low-frequency component exceeds a prescribed amplitude can be obtained as the duration of the high-amplitude low-frequency component. The appearance time of the high-amplitude low-frequency component can be obtained by adding this duration within a prescribed unit time.

The frequency of the high-amplitude low frequency component can be obtained by calculating the number of appearances (the number of times a value constituting an amplitude greater than a certain value is measured for the low frequency waveform) of the waveform constituted by the high-amplitude low frequency component per each prescribed unit time. Further, the strength of the high-amplitude low-frequency component can be obtained by calculating the total of the absolute values of differences between values for high-amplitude low-frequency components for each time occurring within a prescribed unit time and a prescribed amplitude value or the total for the square of the absolute values for these differences.

The strength of the body movement can be obtained by calculating the strength of the high-amplitude low frequency component. Similarly, the frequency of the body movement can be obtained by calculating the frequency of the high-amplitude low frequency component. The appearance time of the body movement can be obtained by adding a prescribed time to the appearance time of the high-amplitude low-frequency component (integrated value). However, it is possible to make the appearance time of the high-amplitude low-frequency component the appearance time of the body movement.

When high-amplitude low-frequency components having amplitudes higher than the prescribed amplitude are extracted using the extraction means for extracting the low-frequency components mentioned previously, the strength, frequency, and duration of this high-amplitude low-frequency component can be calculated using a computer. In this case, the extraction means and the calculation means can be said to be integrated.

Further, the body movement analysis system of the present invention preferably has display means for displaying extracted low-frequency components as a body movement waveform image. The state of the body movement can therefore be easily understood by displaying the low-frequency waveform component extracted as body movement waveform data as a viewable moving-body waveform image.

Well known display means such as a monitor or printer etc. can be used to display that body movement waveform data as a body movement waveform image. For example, an everyday computer monitor or monitor for the measuring means etc. may be used and a body movement waveform image can then be displayed on these monitors. Further, as well as simply displaying a body movement waveform image on a monitor, it is also possible to print and display a body movement waveform image on paper using a printer.

When the extracted low frequency components are composed of digital signals, it is possible to display a body movement waveform image by generating a waveform image from the low frequency components constituted by a digital signal using a computer etc. When the extracted low-frequency component is constructed from an analog signal, this low-frequency component is displayed as is on the monitor.

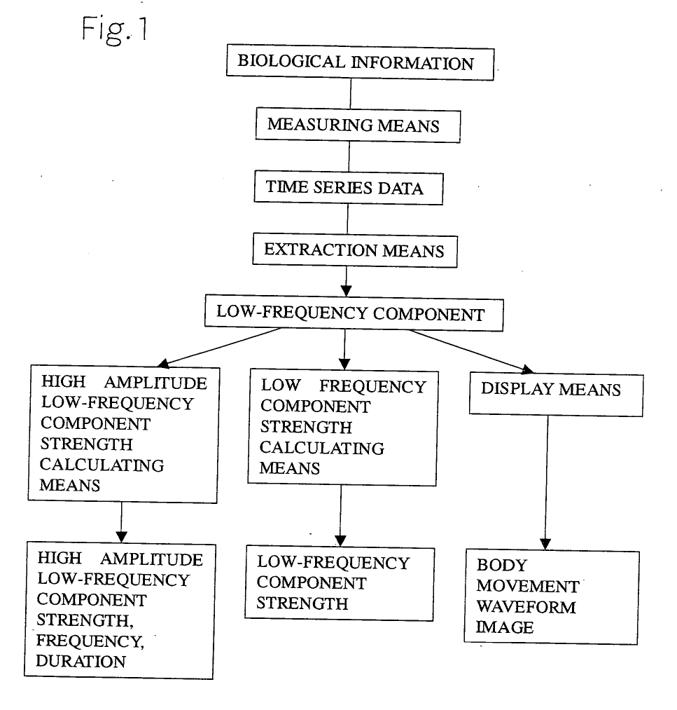

An outline of an embodiment of a body movement analysis system of the present invention is shown in FIG. 1.

Biological information such as movement of respiratory organs or movement of a heart etc. is continuously measured from the body using measuring means such as a respiratory monitor or a heartbeat monitor etc. and time series data is obtained. The obtained time series data is usually constituted by an analog or digital signal (for example, an electrical signal).

A low frequency component of a prescribed frequency or less is then extracted as body movement data from this time series data using extraction means such as a low pass filter or band pass filter, etc. Various information can then be obtained for the body movement by analyzing this body movement waveform data.

The strength of the low frequency component can be calculated from the extracted low-frequency component by low-frequency component strength calculating means such as a computer. High-amplitude low-frequency components having amplitudes of greater than a prescribed amplitude are extracted from the extracted low-frequency components by high-amplitude low-frequency component strength calculating means such as a computer, and at least one or more of the strength, frequency and duration of the high-amplitude low-frequency component is calculated. It is therefore possible to understand body movement of a fixed magnitude or more by calculating one or more of strength, frequency and duration of a high-amplitude low-frequency component having an amplitude of greater than a prescribed amplitude.

Further, it is then possible to display body movement waveform data obtained by extracting low-frequency components from the time series data as a body movement waveform image using display means such as a computer. The state of the body movement can therefore be easily understood by displaying an observable body movement waveform image. Displaying of the body movement waveform image can be displayed on a monitor such as a computer etc. or can be displayed by printing on paper using a printer connected to the computer.

(Body Movement Analysis Method)

The body movement analysis method of the present invention comprises a measurement step for continuously measuring biological information from a body and obtaining time series data, and an extraction step for extracting low frequency components of a prescribed frequency or lower from the time series data as body movement waveform data. The following is a description of the body movement analysis method of the present invention.

Definitions of terminology used in the body movement analysis method of the present invention are the same as body movement analysis systems of the present invention. This description is given in the (body movement analysis system) and is therefore omitted.

The body movement analysis method of the present invention can be implemented using the body movement analysis system of the present invention. A measuring step for continuously measuring biological information from the body and obtaining time series data can be carried out using measuring means for the body analysis system of the present invention. Further, an extraction step for extracting low frequency components of a prescribed frequency or lower from the time series data as body movement waveform data can be carried out using extraction means of the body movement analysis system of the present invention.

The body movement analysis method of the present invention preferably has a low-frequency component strength calculating step for calculating strength of the low-frequency component from the low-frequency component extracted after the extraction step. Similarly, there is further provided a high-amplitude low frequency component strength calculating step for extracting a high-amplitude low-frequency component having a prescribed amplitude or greater from the extracted low frequency component and calculating at least one or more of strength, frequency, and duration of the high-amplitude low-frequency component. Moreover, it is preferable for this body movement analysis method to further comprise a display step for displaying the extracted body movement waveform data as a body movement waveform image.

This low frequency component strength calculating step can be carried out using the low-frequency component strength calculating means of the body movement analysis system of the present invention. This high-amplitude low frequency component strength calculating step can be carried out using the high-amplitude low-frequency component strength calculating means of the body movement analysis system of the present invention. Moreover, the display step can be carried out using the display means of the body movement analysis system of the present invention.

(Embodiments)

The following is a description with reference to the drawings of embodiments employing the body movement analysis system and body movement analysis method of the present invention.

A bedside monitor (Agilent Technologies M1166A) including a heartbeat monitor and a respiratory monitor is used as measuring means for continuously measuring biological information from the body and obtaining time series data. The heartbeat waveform, i.e. an electrocardiogram and respiratory waveform of a new-born baby are measured using these monitors. The electrocardiogram and respiratory waveforms are configured from analog electrical signals. Analog signals are then converted into digital signals using A/D converters (AD Instruments MacLab) built-into the computer. At this time the amplitude range of the heartbeat monitor is set to +/−600 mV. The sampling interval is 0.1 seconds. As a result, time series data (an electrocardiogram) is obtained constructed from sampling values taken continuously every 0.1 seconds. The amplitude range of the respiratory monitor is set to a range of +/−5V Similarly, time series data is obtained continuously every 0.1 seconds by the A/D converter.

Further, time series data for the electrocardiogram and the respiratory waveform converted to a digital signal by this MacLab is inputted to the computer (Macintosh produced by Apple computer). A low frequency component for 1 Hertz or less and a low-frequency component for 0.5 Hertz or less are then extracted for the cardiograph and respiratory waveform, respectively, by this computer. In this embodiment, the extraction means for extracting the low-frequency component can be implemented using a computer. The electrocardiogram, respiratory waveform and extracted low-frequency component shown here are outputted from a printer using this computer. Namely, the computer and the printer are configured from display means.

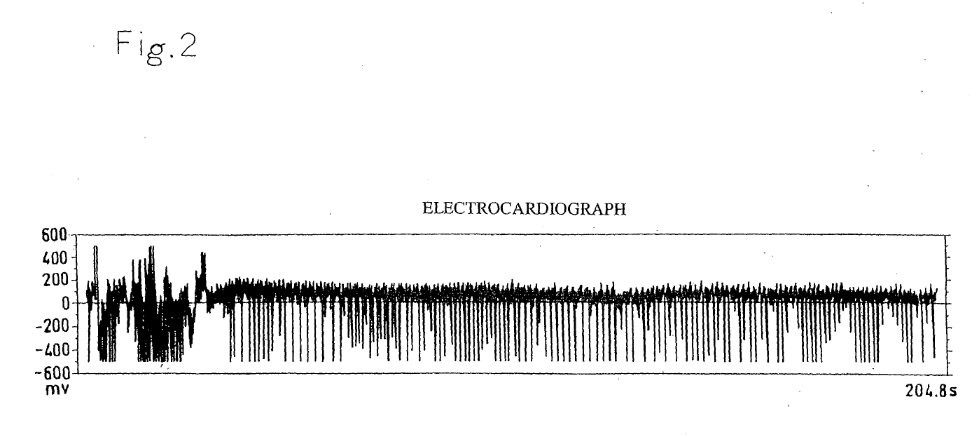

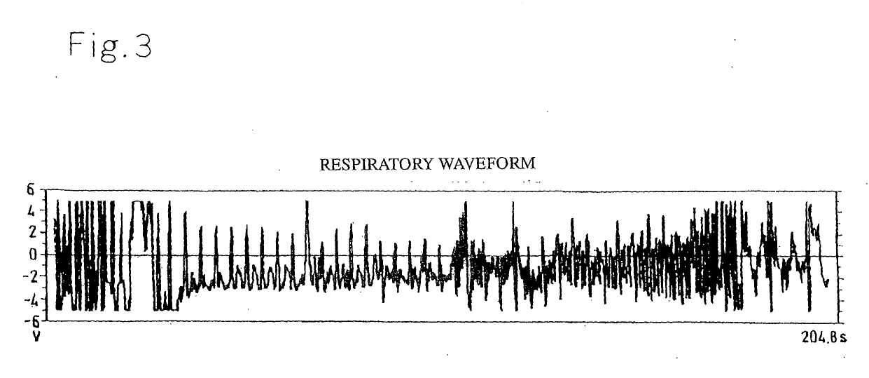

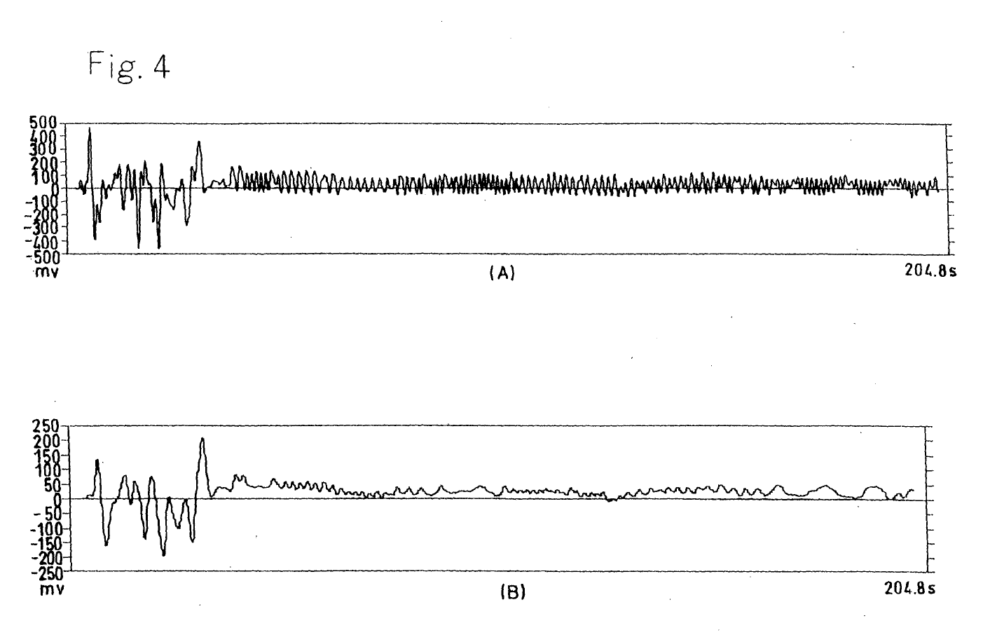

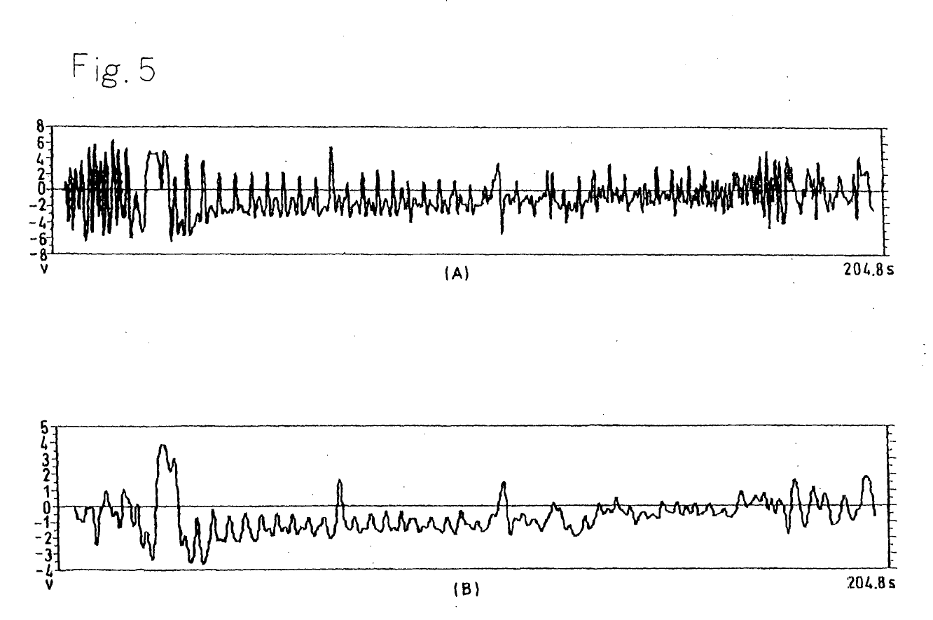

An electrocardiogram is shown in FIG. 2 and a respiratory waveform is shown in FIG. 3. A waveform for a low-frequency component of 1 Hertz or less extracted from the electrocardiogram using a computer is shown in FIG. 4(A) as a body movement waveform image and a waveform for a low-frequency component of 0.5 Hertz or less is shown in FIG. 4(B) as a body movement waveform image. A waveform for a low-frequency component of 1 Hertz or less extracted from the respiratory waveform using a MacLab is shown in FIG. 5(A) as a body movement waveform image and a waveform for a low-frequency component of 0.5 Hertz or less is shown in FIG. 5(B) as a body movement waveform image.

It is then possible to understand the state of the body movement by extracting a low-frequency component of 1 Hertz or less and a low-frequency component of 0.5 Hertz or less and displaying this as a body movement image. Larger movements in body movement can be more clearly understood from the low-frequency component for 0.5 Hertz or less than from the low-frequency component for 1 Hertz or less.

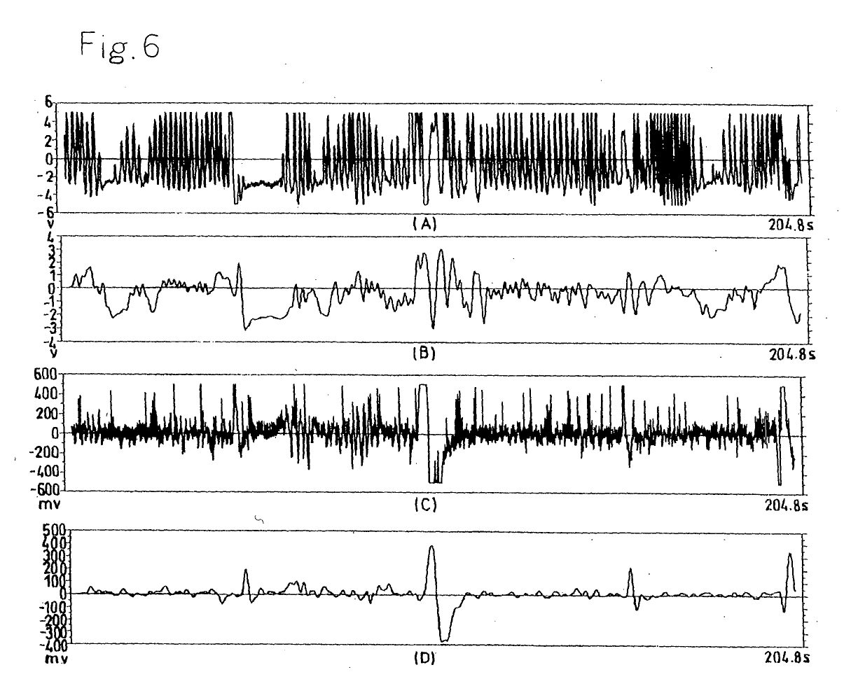

Respiratory waveforms occurring in the same time band outputted from the computer are shown in FIG. 6(A) and waveforms for low-frequency components of less than 0.5 Hertz extracted from the respiratory waveform are shown in FIG. 6(B). Further, an electrocardiogram is shown in FIG. 6(C) and a waveform for a low-frequency component of 0.5 Hertz or less extracted from this cardiograph is shown in FIG. 6(D). Comparing the waveforms of FIG. 6(B) and FIG. 6(D), it can be confirmed that there are differences with regards to phase differences and amplitude but it can be understood that the same kind of waveform is shown. It can therefore be understood that it is possible to understand body movement by extracting a low-frequency component in the same way for either the cardiograph or the respiratory waveform.

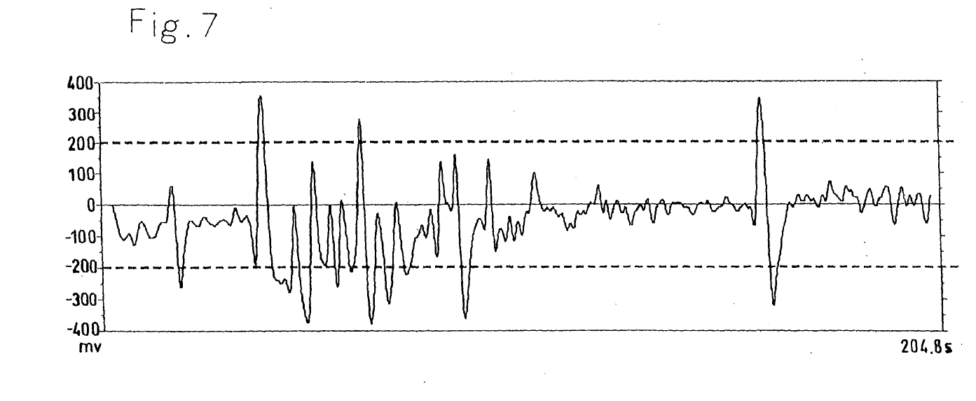

FIG. 7 shows a waveform for a low-frequency component extracted from a cardiograph obtained by measuring at 0.1 second intervals in an amplitude range of +/−600 mV. A high-amplitude low-frequency component of an amplitude of greater than 200 mV and less than −200 mV is calculated and taken to be the body movement. The lines for 200 mV and −200 nV are shown in FIG. 7 using dashed lines. When it is wished to only extract portions for the line of the waveform above the 200 mV line and portions for the line for the waveform for less than −200 mV, i.e. only substantial movements of the body as body movement, then it is possible to extract a high-amplitude low-frequency component of a fixed amplitude or greater from the low-frequency component.

By extracting a high-amplitude low-frequency component of a fixed amplitude or greater to catch body movement, the strength, frequency and duration for body movement of a fixed size or greater can be calculated.

Further, a time with 0.5 seconds either side of the sensing of the high-amplitude low-frequency component is added to the time for which this high-amplitude low-frequency component continues for. An appearance time per unit time as described later is then obtained from this calculated time. These calculations can be implemented using a computer.

In this embodiment, the following symptoms are shown for a new born-baby for which biological information is measured. The respiratory condition is stable at the time of birth. Apnea is then confirmed after crying, but the apnea then gradually reduces. At four days old, the animation has disappeared, and at five days old a light rash appears, which is diagnosed as new-born exanthematous. At four days old, it is confirmed from the respiration waveform that respiration becomes short and fast.

A low-frequency waveform component of 0.5 Hertz or less for a continuous eight hours is then extracted from the electrocardiogram measured continuously using a heartbeat monitor on the newly born baby. Further, that for which the absolute value of the amplitude is 200 mV or greater is then extracted from the extracted low-frequency component. The average appearance time (the meaning of this is as described previously) for the body movement for thirty-minute units occurring in an eight hour period is then calculated. The units of the average appearance time for which the body movement appears (thirty minute units) is calculated at one day old, and two units are calculated for different time bands for two days old. One unit is then calculated for three days old, and one unit is calculated for four days old (at midnight). The average appearance time of the body movement in one unit for one day old is 1333 (measured at 0.1 second intervals. The same goes for the following. The time is therefore 1333×0.1 seconds=133.3 seconds). The average appearance time for body movement at two days old is 1337 (0.1 seconds) for the previous time band and 3560 (0.1 seconds) for the following time band, the average appearance time for body movement at three days old is 3495 (0.1 seconds), and the average appearance time for body movement at four days old is 635 (0.1 seconds).

At four days old, it is clear that the duration of the body movement is becoming shorter. It can be understood that the animation has disappeared at four days old from the time of appearance of the body movement. Further, by comparing the appearance time of the body movement, it is possible to achieve early diagnosis of infection in a newly born baby.

Further, it is also possible to more fully understand the condition of a patient by comparing and analyzing a respiratory wave and electrocardiogram extracted at the same time.

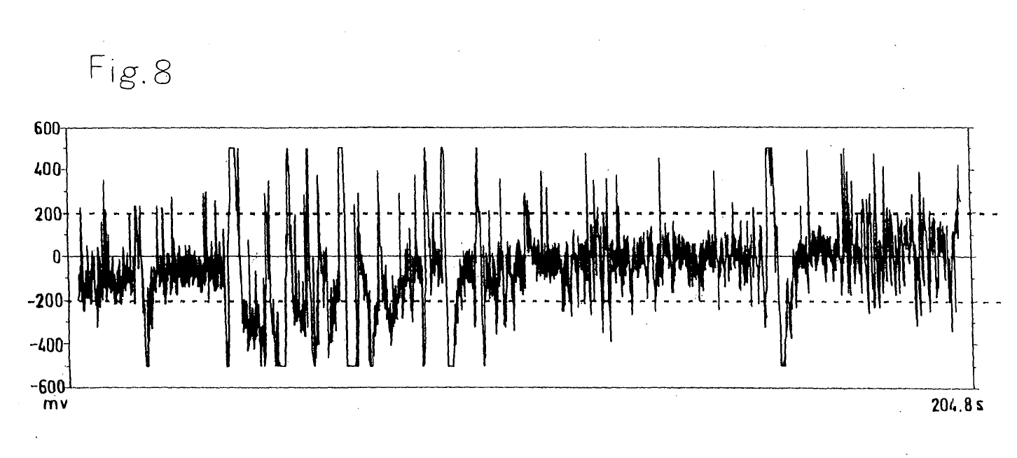

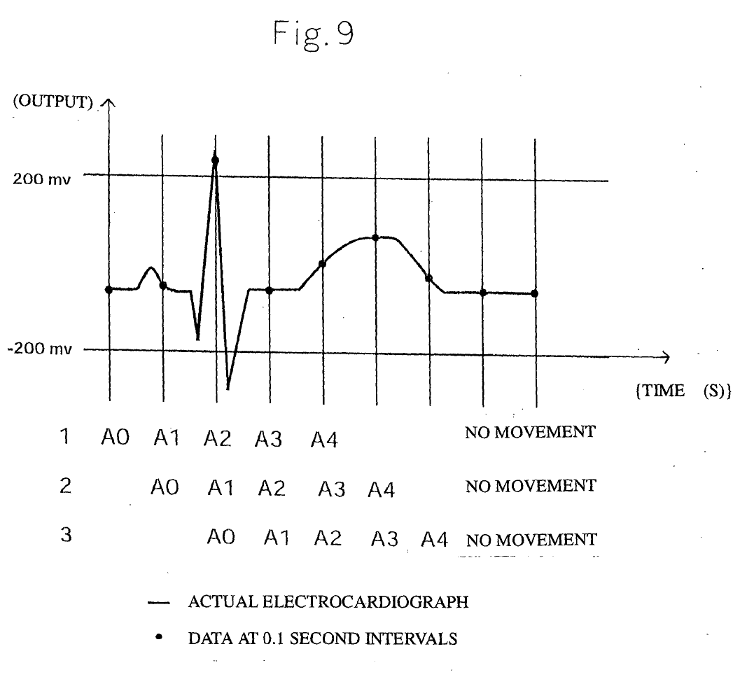

Extraction of the high-amplitude low-frequency component can also be carried out as described below. In FIG. 8 and FIG. 9, an electrocardiogram is shown where measurements are taken at 0.1 second intervals for an amplitude range of +/−600 mV. FIG. 9 is an enlarged outline view of the essential parts of FIG. 8. In this case, the continuous time series data is continuously replaced in the order AO, A1, A2, A3, A4. The sections A0 to A4 constitute a total of four seconds. When at least four of the items of data substituted for AO, A1, A2, A3 and A4 are values of 200 mV or more, or at least four points of the frequency components for AO, A1, A2, A3 and A4 are values of −200 mV or less, and the time series data (frequency component) assigned with the number for AO is taken as the high-amplitude low-frequency component. The numbers for AO, A1, A2, A3 and A4 are sequentially shifted one at a time, with AO being substituted for the value at A1, and A1 being substituted with the value at A2. The next value at the continuous data for the time series is then substituted at A4. In this manner, the same operation for deciding the high-amplitude low-frequency component is repeated and the high-amplitude low-frequency component is extracted.

This is to say that a QRS wave constituted by a high-amplitude spike is included in the electrocardiogram, and it is necessary for this to be removed to provide differentiation from the basic wave. This QRS wave has a frequency of five Hertz or more and has upper and lower QRS wave vertexes within 0.2 seconds. A hole is therefore provided at one section of the five points within 0.4 seconds. If the QRS wave is then inserted into this hole, then this can be ignored as it is spike occurring within 0.2 seconds bearing no relationship to the basic wave. In this case, it is possible to regard the body movement by extracting the high-amplitude component for a certain fixed time while providing a hole at a certain fixed section while eliminating the QRS wave component.

Further, a time with 0.5 seconds either side of the sensing of the high-amplitude low-frequency component is added to the time for which this high-amplitude low-frequency component continues for. An appearance time per unit time as described later is then obtained from this calculated time. These calculations can be implemented using a computer.

The present invention is therefore capable of extracting the high-amplitude low-frequency component in one step and performing calculations. In this case, the extraction means extracts the high-amplitude low-frequency component.

In this method, the time of appearance of the body movement can be calculated in the same manner from the same electrocardiogram as for the new born baby described above. Namely, the average appearance time for the body movement for thirty-minute units occurring in an eight hour period is then calculated. The average appearance time of the body movement for one day old is 2402 (measured at 0.1 second intervals. The time is therefore 2402×0.1 seconds=240.2 seconds). The average appearance time for body movement at two days old is 2409 (0.1 seconds) for the previous time band and 5237 (0.1 seconds) for the following time band, the average appearance time for body movement at three days old is 5258 (0.1 seconds), and the average appearance time for body movement at four days old is 1090 (0.1 seconds). Even when the appearance time for the body movement is calculated using this method, it can be understood that the body movement appearance time becomes dramatically smaller at four days old.

### INDUSTRIAL APPLICABILITY

The body movement analysis system and body movement analysis method of the present invention are capable of extracting body movement waveform data from time series data obtained by continuously measuring biological information from a body. It is therefore possible to obtain various information relating to the body movement using this body movement waveform data.

The body movement analysis system and the body movement analysis method of the present invention is therefore capable of obtaining body movement information such as strength, frequency and appearance time for body movement of greater than a prescribed size from a low-frequency component extracted as the body movement waveform data.

The body movement analysis system of the present invention are capable of extracting body movement waveform data from time series data obtained by continuously measuring biological information from a body and therefore has time series data for specific organs and parts of the body. It is therefore possible for the extracted body movement waveform data and this time series data to be provided as data capable of enabling comparison of the condition of specific organs or parts of the body and the condition of the body movement.

1. A body movement analysis system comprising measuring means for continuously measuring biological information from a body and obtaining time series data; and extraction means for extracting low frequency components of a prescribed frequency or lower from the time series data as body movement waveform data.

2. The body movement analysis system as disclosed in claim 1, further comprising low-frequency component strength calculating means for calculating strength of the low-frequency component from the low-frequency component.

3. The body movement analysis system as disclosed in claim 1 or claim 2, further comprising high-amplitude low frequency component strength calculating means for extracting a high-amplitude low-frequency component having a prescribed amplitude or greater from the low frequency component and calculating at least one or more of strength, frequency, and duration of the high-amplitude low-frequency component.

4. The body movement analysis system as disclosed in claim 1, 2, or 3, further comprising display means for displaying extracted body movement waveform data as a body movement waveform image.

5. The body movement analysis system as disclosed in claim 1, 2, 3, or 4, wherein the measuring means is a heartbeat monitor.

6. The body movement analysis system as disclosed in claim 1, 2, 3, or 4, wherein the measuring means is a respiratory monitor.

7. The body movement analysis system as disclosed in claim 1, 2, 3, 4, 5 or 6, wherein the body is the body of a person.

8. The body movement analysis system as disclosed in claim 7, wherein the prescribed frequency is 0.5 Hertz.

9. A body movement analysis method comprising a measurement step for continuously measuring biological information from a body and obtaining time series data; and an extraction step for extracting low frequency components of a prescribed frequency or lower from the time series data as body movement waveform data.

10. The body movement analysis method as disclosed in claim 9, further comprising a low-frequency component strength calculating step for calculating strength of the low-frequency component from the low-frequency component.

11. The body movement analysis method as disclosed in claim 9 or claim 10, further comprising a high-amplitude low frequency component strength calculating step for extracting a high-amplitude low-frequency component having a prescribed amplitude or greater from the low frequency component and calculating at least one or more of strength, frequency, and duration of the high-amplitude low-frequency component.

12. The body movement analysis system as disclosed in claim 9, 10, or 11, further comprising a display step for displaying extracted body movement waveform data as a body movement waveform image.
